(19) United States
(12) Patent Application Publication
(10) Pub. No.: US 2008/0172757 A1
(43) Pub. Date: Jul. 17, 2008

(54) Seed Stock

(75) Inventors: Rahel Blatter, Monthey (CH); Urban Anderau, Monthey (CH); Charly Bressoud, Vionnaz (CH); Helge Sierotzki, Zumikon (CH); Rita Stalder, Magden (CH)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: SYNGENTA PARTICIPATIONS AG

(21) Appl. No.: 11/912,026

(22) PCT Filed: Apr. 20, 2006
(86) PCT No.: PCT/EP06/03642

§ 371 Date: Mar. 25, 2008

(30) Foreign Application Priority Data
Apr. 22, 2005 (DE) 10-2005-018-862.1

(51) Int. Cl. A01H 1/02; A01H 5/00

(52) U.S. Cl. 800/265; 800/317.2

(57) ABSTRACT

The present invention relates to improved potato seed and propagation stock which has different resistance characteristics with respect to different pathogens, in particular however to fungi of the Phytophthora genus.

A further subject matter of the invention relates to a method for the hybridisation of Phytophthora resistance into different potato culture forms.

The present invention relates to improved potato seed and propagation stock which has different resistance and tolerance characteristics with respect to different pathogens, in particular however outstanding resistance to fungi of the Phytophthora genus, and preferably here to Phytophthora infestans.

A further subject matter of the invention relates to a method for the hybridisation of Phytophthora resistance into different potato culture forms. The potato belongs to the Solanaceae family (nightshade family). It propagates vegetatively by offshoot tubers or generatively by forming seeds. Potatoes can be divided into two groups: Solanum tuberosum spp. tuberosum and Solanum tuberosum spp. andigena.

The herbaceous plant has white to purple blossom and can grow to a height of a metre. Potato leaves are imparipinnate.

The potato has 12 chromosomes in the ploidy grades diploid (2n) or tetraploid (4n).

The domesticated types are autotetraploid and, unlike the diploid wild types, can self pollinate. They show tetrasomic heredity.

Nowadays, the potato is also still one of the most important suppliers of carbohydrate to man and so is one of the basic foods. In 2000 overall production worldwide stood at 329 million tonnes. The main producers are China and the combined states of the previous Soviet Union.

The success of the potato is also dependent upon the plant's modest demands upon its environment. It can thrive at heights of up to 2000 metres above sea level, on the edge of polar regions or in tropical environments.

The greatest problem affecting the potato industry worldwide is leaf and tuber blight as a result of Phytophthora infestans infections. Moreover, various virus diseases, triggered e.g. by the Potato Leaf Roll Virus (PLRV), the Potato Virus X (PVX) and the Potato Virus Y (PVY), as well as grub damage caused by the potato beetle (Leptinotarsa decemlineata) pose a problem.

The potato virus diseases can be largely controlled in the industrial nations by seed certification and the use of insecticides to counter the virus disease carriers (aphids). The same applies to the potato beetle which can also be successfully combated by the use of insecticides.

However, by far the greatest problem economically, because up to now it remains largely unsolved, is posed by leaf and tuber blight caused by infection with Phytophthora infestans. This occurs as an epidemic, and an early, strong infestation can lead to a total loss of the crop. In the eighties and nineties the fungus caused losses amounting to hundreds of millions in North America alone. The “International Potato Center” in Lima estimates that crop losses which are due to P. infestans infections amount to approximately 15% of the world potato production.

Phytophthora infestans belongs to the oomycetes and, as well as the potato, uses other nightshade varieties as a host plant, such as e.g. the tomato. In the course of a typical infection the lower leaves of the plant are first of all infested, followed by the leaf stalks and stems, and finally also the tubers. The typical grey-white fungi form on the lower side of the leaves from where the spores are released. The spores spread from a few primary sources within a short time, in particular in humid atmospheric conditions, and are capable of covering large distances. A whole potato field can thus be totally infested within just a few days.

Up till now the pest could only be propagated asexually in Europe and the USA. Since the infiltration of the sexual form the development of even more virulent genotypes is being promoted by the increased recombination possibilities, and this can consequently lead to extensive crop losses. Correspondingly, the adaptation times to potato varieties which have vertical resistance are greatly shortened.

In conventional agriculture leaf and tuber blight is mostly combated with the help of fungicides. However, since the eighties Phytophthora strains have been in existence which are increasingly resistant to the commonly used fungicides. It is therefore an urgent objective to develop alternatives which guarantee effective control of leaf and tuber blight.

One of these possible alternatives, the cultivation of potatoes resistant to Phytophthora infestans, has proven to be extremely difficult because only horizontal resistances are long-lasting and the cultivation process is very long-winded. The research in this field is being carried out in particular by the “International Potato Center”, radical success not yet having been achieved however.

A further possibility is the use of genetic strategies for generating resistance. In principle it is possible to generate vertical resistance, but this is not long-lasting and so is not very efficient. On the other hand, it has not yet been possible to implement horizontal resistance with the help of genetic methods.

Within the framework of the present invention a potato plant is now provided for the first time which has outstanding and lasting resistance to Phytophthora infestans infections.

Within the framework of the present invention, resistance is to be understood in particular as meaning horizontal resistance which in conventional language use also means general, non-specific or quantitative resistance which enables the plant to prevent or at least to limit infestation and colonisation by all known strains or genera of pest. A limited infestation or a limited colonisation is to be understood as meaning, for example, as an infection pattern of Phytophthora infestans which is interrupted in that the formation of mycelium on the lower side of the leaf and sporulation are prevented.

Unlike vertical resistance which only offers protection against specific genera or strains of the pest and which can be prevented relatively easily, horizontal resistance offers protection against the pest which has no time-limit and which is therefore robust.

In one specific embodiment of the invention a resistant plant is to be understood as meaning a potato plant which remains totally symptom-free following infection with the pest.

Therefore, the present invention relates to a potato plant which is resistant to Phytophthora infestans infections.

In particular, the invention relates to a potato plant which has horizontal, and so outstanding and lasting resistance to Phytophthora infestans infections and preferably remains free of symptoms, in particular however resistance to infections caused by pathotypes which have at least one, but in particular two, three, four, five, six, seven, eight, nine, ten, or in particular all of the known virulence genes V1-V11 in different combinations.

In one specific embodiment of the present invention a potato plant is provided which has horizontal, and so outstanding and lasting resistance to Phytophthora infestans infections and preferably remains free of symptoms, in particular however resistance to infections which are caused by one or more of the following pathotypes, in particular however by all of the following pathotypes which have at least 2 of the known virulence genes V1-V11 in the following combination:
Pathotype 1: with virulence genes 3, 4, 5, 7 and 9
Pathotype 2: with virulence genes 1, 3, 4, 7, 8, 9, 10 and 11
Pathotype 3: with virulence genes 2 and 7
Pathotype 4: with virulence genes 3, 4, 5 and 11
Pathotype 5: with virulence genes 1, 3, 4, 7

The aforementioned pathotypes 1-5 can be determined, for example, with the so-called DPL (Dutch Potato Lines) test system, a test system consisting of 11 potato plants of the Bintje variety, each plant of the system having an R gene corresponding to the above-specified V genes.

In a further embodiment of the present invention a potato plant is provided which has horizontal, and so outstanding and lasting resistance to Phytophthora infestans infections, and which preferably remains free of symptoms, in particular however of infections caused by a strain mixture comprising 230 isolates according to I. Irzhansky and Y. Cohen, 2006 (“inheritance of resistance against Phytophthora infestans in Lycopersicon pimpenellifolium L3707”, accepted for publication in Euphytica).

Furthermore, the invention relates to a potato plant which has horizontal, and so outstanding and lasting resistance to Phytophthora infestans infections and which preferably remains free of symptoms, in particular however to infections caused by

(a) one or more of the following pathotypes, in particular however by all of the following pathotypes which have at least 2 of the known virulence genes V1-V11 in the following combinations:
Pathotype 1: with virulence genes 3, 4, 5, 7 and 9
Pathotype 2: with virulence genes 1, 3, 4, 7, 8, 9, 10 and 11
Pathotype 3: with virulence genes 2 and 7
Pathotype 4: with virulence genes 3, 4, 5 and 11
Pathotype 5: with virulence genes 1, 3, 4, 7; and

(b) a strain mixture comprising 230 isolates according to I. Irzhansky and Y. Cohen, 2006 (“Inheritance of resistance against Phytophthora infestans in Lycopersicon pimpenellifolium L3707”, accepted for publication in Euphytica).

In particular, the invention relates to a potato plant which has horizontal, and so outstanding and lasting resistance to Phytophthora infestans infections, and which preferably remains free of symptoms, characterised in that the resistance feature can be obtained by hybridising a precursor plant which has the genetic form of SCP-02 (DSM 17671).

The invention further relates to a potato plant which has horizontal, and so outstanding and lasting resistance to Phytophthora infestans infections, and which preferably remains free of symptoms, hybridised from a precursor plant which has the genetic form of SCP-02 (DSM 17671).

In one specific embodiment of the invention a potato plant is provided which has horizontal, and so outstanding and lasting resistance to Phytophthora infestans infections and the genetic form of SCP-02 (DSM 17671).

Also included is a potato plant wherein the aforementioned Phytophthora resistance feature is characterised in that the natural infection pattern of Phytophthora infestans is interrupted in that the formation of mycelium on the lower side of the leaf and sporulation are prevented. This feature characteristic is found in particular with infection with a highly pathogenic Phytophthora strain such as, for example, strain 90128.

Moreover, the invention relates to a method for hybridising resistance, in particular however horizontal, and so outstanding and lasting resistance to Phytophthora infestans infections which preferably leads to potato plants which remain free of symptoms in potato plants which do not have this resistance, characterised in that
a) a potato plant which has the genetic form of SCP-02, and so resistance to Phytophthora infestans infections, a plant from which a sample in the form of a meristem culture has been deposited at the German Collection of Microorganisms and Cell Cultures GmbH (DSMZ) in 38124 Braunschweig, Mascheroder Weg 1b, dated 22 Apr. 2005 under deposit number DSM 17671, is crossed with a potato plant which is susceptible to Phytophthora infestans infections;
b) appropriate measures are taken so as to avoid mixed pollination;
c) seeds are isolated from the crop ripe fruits and sown;
d) plantlets are raised, and these are tested in an appropriate test system for resistance to Phytophthora infestans;
e) plants are selected which have the feature of resistance to Phytophthora infestans infections.

In one particular embodiment of this method the F1 hybrids resulting from the crossing which have the feature of resistance to Phytophthora infestans infections are re-crossed between one and more times with a parent, preferably however with the Phytophthora sensitive parent.

Also included by the present invention is a method for producing a potato plant which has resistance to Phytophthora infections, in particular however horizontal and so outstanding and lasting resistance to Phytophthora infestans infections, which preferably leads to potato plants which remain free of symptoms, characterised in that
a) a potato plant which has the genetic form of SCP-02 and so resistance to Phytophthora infestans infections, a plant from which a sample in the form of a meristem culture has been deposited at the German Collection of Microorganisms and Cell Cultures GmbH (DSMZ) in 38124 Braunschweig, Mascheroder Weg 1b, dated 22 Apr. 2005 under deposit number DSM 17671, is crossed with a potato plant which is susceptible to Phytophthora infestans infections;
b) appropriate measures are taken so as to avoid mixed pollination both during and after the artificially implemented pollination process;
c) seeds of the F1 generation are isolated from the crop ripe fruits and sown;
d) plantlets are raised, and these are tested for resistance to Phytophthora infestans;
e) Phytophthora-resistant plants are re-crossed between one and more times with the Phytophthora-susceptible parent;
f) in each re-crossed generation obtained in this way, the plants are tested for the presence or absence of the resistance feature,
g) a plant is raised which is characterised by the desired feature of resistance to Phytophthora infections.

Since the potato is capable of self-pollination, in order to be able to carry out the cross or combination cultivation methods successfully, controlled and targeted process management is required which guarantees the transfer of pollen from the paternal parent to the mother plant, at the same time excluding any undesired mixed pollination.

This necessitates the use of technical measures such as e.g. the manual cutting of the anther out of the flower of the mother plant as well as the specific transfer of pollen from the paternal parent to the stigma of the mother plant. Next, further measures must be taken in order to prevent mixed pollination of the previously artificially pollinated flower, such as e.g. by placing the pollinated flower in a bag.

Alternatively, a sterile parent can also be used as a crossing partner, and this renders emasculation futile.

Further measures to supplement the method described above relate to the specific induction of blossom by appropriate measures described in detail in the examples and to the stimulation of seed germination.

The formation of blossom with potato plants can be induced, for example, by continuously removing the tubers that form from the plant. In so doing the tubers which form are carefully removed from the plant every 3-8 weeks, in particular however every 4-6 weeks, and most particularly every 4-5 weeks without damaging the latter, and in this way the plant is stimulated to form more abundant blossom.

Seed germination can be improved by incubating the seeds in the crop ripe seed vessel after the latter has been removed from the plant and has preferably been sliced open for a period of between 8 hours and 4 days, in particular of between 12 hours and 3 days, but preferably of between 1 and 2 days. Alternatively to this, the incubation can also take place for the specified period in an extract taken from the seed vessels.

The F1 generation potato plants which can be obtained within the framework of the method according to the invention can be obtained in the form of tubers or by meristem culture and be propagated.

Testing of the progeny for resistance feature characteristics can be implemented with the help of leaf segments and/or tuber slices which are prepared appropriately for the tests, e.g. by incubation in petri dishes and subsequent inoculation with spore suspensions of the different test strains and pathotypes.

For the first time, the present invention provides a potato plant which has horizontal, and so outstanding and lasting resistance to Phytophthora infestans infections, in particular however infections caused by Phytophthora pathotypes which contain at least one or more of the virulence genes selected from the group of virulence genes 1, 2, 3, 4, 5, 7, 8, 9, 10 and 11 in different combinations.

In one specific embodiment of the invention a potato plant is made available which has horizontal, and so outstanding and lasting resistance to Phytophthora infestans pathotypes which contain two, three, four, five, six, seven, eight, nine, ten or in particular all of the virulence genes selected from the group of virulence genes 1, 2, 3, 4, 5, 7, 8, 9, 10 and 11 in different combinations.

Therefore, the present invention relates in particular to a potato plant which has outstanding and lasting resistance to Phytophthora infestans infections, in particular however to infections which are caused by one or more of the following pathotypes, in particular however by all of the following pathotypes which have at least 2 of the known virulence genes V1-V11 in the following combinations:
Pathotype 1: with virulence genes 3, 4, 5, 7 and 9
Pathotype 2: with virulence genes 1, 3, 4, 7, 8, 9, 10 and 11
Pathotype 3: with virulence genes 2 and 7
Pathotype 4: with virulence genes 3, 4, 5 and 11
Pathotype 5: with virulence genes 1, 3, 4, 7.

The invention relates in particular to a potato plant which can be obtained by hybridising the resistance feature of a precursor plant which has the genetic form of SCP-02, from which a sample in the form of a meristem culture has been deposited at the German Collection of Microorganisms and Cell Cultures GmbH (DSMZ) in 38124 Braunschweig, Mascheroder Weg 1b, dated 22 Apr. 2005 under deposit number DSM 17671.

The aforementioned SCP-02 potato plant has a white skin and has a flavour comparable to that of the commercial varieties “Matilda” and “Bintje”.

In cold storage (5° C.), from January onwards the tubers easily produce sprouts, and from March onwards clear seedling growth begins which is slightly more pronounced than with the comparable variety “Bintje”.

The planted out tubers produce the first sprout tips on the surface in a comparable time frame to the comparable variety “Bintje”.

The development time leading up to flowering is also identical to that of “Bintje”. However, the plant size is considerably smaller than with this standard variety.

The blossom of SCP-02 is white, the flowers adhere well to the bush and consequently develop fruits without any problem.

Storability is also normal (until March). When using germination retardants storage possibilities are implementable similar to with the comparable variety “Bintje”.

Notwithstanding the resistance to Phytophthora infections both of the leaf and of the tuber, the plant shows average to high susceptibility to viruses such as e.g. the leaf roll virus and average susceptibility to Alternaria infections. The attractiveness of the plants for aphids is average, and less for potato beetles.

Potato plants according to the present invention which can be obtained by the hybridisation of the resistance feature of a precursor plant which has the genetic form SCP-02 are therefore characterised in particular by the feature of resistance to Phytophthora infestans infections, in particular however to infections which are triggered by the Phytophthora pathotypes selected from the group consisting of strain 96; strain 90128, strain 91002, strain 91011; and strain 88069S, the aforementioned feature being freely transferable to the progeny of the aforementioned potato plant within the framework of crossing tests. Here, the heredity of the aforementioned feature to the progeny plants follows the laws of dominant or semi-dominant heredity.

Within the framework of this invention a resistant potato plant is to be understood as meaning a plant which, with artificial inoculation of the leaf or tuber with a spore suspension of Phytophthora strains which cover the majority of R-genes of the DPL such as e g. strain 96; strain 91002, strain 91011; and strain 88069S, in a spore density of 50,000 spores/ml, shows no symptoms typical of a Phytophthora infection or with which, in so far as such symptoms can be identified, such as e.g. in the case of use of highly pathogenic Phytophthora strains such as e.g. strain 90128, at least no mycelium growth on the leaf or, in the case of a tuber infection, only an atypical, ball-shaped mycelium growth can be detected, but under no circumstances sporulation of the fungus. Therefore, any infection symptoms occurring locally remain restricted to the place of infection without, however, the infection spreading over the whole plant or without it being possible for the infection to be carried further onto other plants.

A further subject matter of the invention relates to a potato plant wherein the aforementioned resistance feature is characterised in that the natural infection pattern of Phytophthora infestans is interrupted so that the formation of mycelium on the leaf of the infected plant and sporulation are prevented.

In particular, the invention relates to potato plants which can be obtained by hybridising the resistance feature of a precursor plant which has the genetic form of SCP-02, wherein the aforementioned resistance feature is characterised in that the natural infection pattern of Phytophthora infestans is interrupted so that mycelium formation on the leaf of the infected plant and sporulation are prevented.

The present invention further relates to a method for hybridising the feature of resistance to Phytophthora infestans infections, and this essentially comprises the following steps: (a) providing a potato plant which has resistance to Phytophthora infestans infections, such as e.g. a potato plant which has the genetic form of SCP-02 and so the aforementioned resistance, from which a sample in the form of a meristem culture has been deposited at the German Collection of Microorganisms and Cell Cultures GmbH (DSMZ) in 38124 Braunschweig, Mascheroder Weg 1b, dated 22 Apr. 2005 under deposit number DSM 17671, (b) crossing this plant with another potato plant which is susceptible to infections with Phytophthora infestans, (c) producing F1 hybrid plants and (d) selecting a hybrid plant which has resistance to Phytophthora infestans infections.

In order to combine the desirable properties of two parent plants with one another, so-called cross or combination cultivation can be implemented. Here the F₁ generation plants, which all look the same, are crossed once again. Only in the F₂ generation are the features split, and one can see which plants have the desired combination. This is the case with max. one quarter of the plants. Cultivation is then continued with these plants until after approx. 7-8 generations the division numbers are so small that a new variety with the desired combination of features has been created. Cross cultivation is still the most frequently used cultivation method for potatoes.

If it is the aim of the cultivation programme to hybridise a selected feature such as, e.g. resistance, into an otherwise already optimised high performance variety with desired properties, so-called re-cross cultivation is offered as a variation of this cross or combination cultivation. Here, the F1 progeny is re-crossed between one and more times with the parent plants which have the desired properties, generally therefore with the high performance variety. It is the aim of this re-crossing to reproduce the genotype of the high performance variety as completely as possible with the exception of one or more genes which are responsible for the characteristics of the selected feature. The re-crossing is a form of inbreeding, it being automatically guaranteed that as the number of re-crossing steps increases, the properties of the high performance variety are successively reproduced. The only selection criterion used here relates to the selected feature, e.g. resistance, which can generally be demonstrated easily by means of appropriate test methods. The number of re-crossing steps is generally between 1 and 4, or more, depending upon how completely the genotype of the high performance variety is to be reproduced.

Within the framework of the present invention e.g. a potato plant which has the genetic form of SCP-02 and so resistance to Phytophthora infestans infections, a sample from which in the form of a meristem culture has been deposited at the German Collection of Microorganisms and Cell Cultures GmbH (DSMZ) in 38124 Braunschweig, Mascheroder Weg 1b, dated 22 Apr. 2005 under deposit number DSM 17671, can within the framework of a re-crossing programme first of all be crossed with a Phytophthora-sensitive potato plant which has desirable properties, such as e.g. a high performance variety, which is then used as the parent repeatedly used for re-crossing (recurrent parent). With this type of crossing, the resistant parent can serve either as the female or as the male crossing partner.

The resulting F1 plant is then re-crossed between one and more times with the Phytophthora-sensitive parent plant, such as e.g. one of the high-performance varieties (recurrent parent) so as to further reduce the genome portion of the resistant parent plant and to successively replace it by the genome of the Phytophthora-sensitive parent plant. The aim is to obtain a plant which has between 80% and 99.5%, better between 90% and 99%, in particular however between 95% and 98% of the genome of the sensitive parent plant that otherwise however is provided with desirable properties (such as e.g. a high-performance variety), but which moreover is still characterised by the feature of resistance. In each re-crossing generation obtained in this way, the plants must be tested for the presence or absence of the resistance feature. In the case of resistance this can be implemented very easily by using the test methods described in detail in the examples.

After the final re-crossing, self-fertilisation is preferably carried out.

Since the potato is capable of self-pollination, in order to be able to carry out the cross or combination cultivation methods successfully, controlled and specific process management is required which guarantees the transfer of pollen from the paternal parent to the mother plant, at the same time excluding undesirable mixed pollinations.

This makes it necessary to use technical measures such as e.g. manually cutting out the anther from the flower of the mother plant and the specific transfer of pollen from the paternal parent to the stigma of the mother plant. Next, further steps must be taken in order to prevent mixed pollinations of the previously artificially pollinated flower, such as e.g. by placing the pollinated flower in a bag.

Since the germination rate of potato seeds is often only low, in particular in the case of high-performance varieties, this must, if appropriate, be increased by using appropriate measures. One possible measure here is to leave the seeds here after the fruit has been sliced open for a period of between a few hours and 4 days, in particular however for a period of between 6 hours and 3 days, or for a period of between 12 hours and 2 days, in particular for a period of between 1 and 2 days before the sowing then take place in an appropriate culture medium. Alternatively the incubation of the isolated seeds can then also take place over the aforementioned period of time in a plant extract which can be extracted from the ripe potato fruits.

Therefore, the present invention further relates to a method for the hybridisation of resistance to infections with Phytophthora infestans in potato plants which do not have this resistance, characterised in that
a) if appropriate, the potato plant provided as the mother plant is stimulated by appropriate measures to form blossom;
b) a potato plant which has the genetic form of SCP-02 and so resistance to Phytophthora infestans infections, a plant from which a sample in the form of a meristem culture has been deposited at the German Collection of Microorganisms and Cell Cultures GmbH (DSMZ) in 38124 Braunschweig, Mascheroder Weg 1b, dated 22 Apr. 2005 under deposit number DSM 17671, is crossed with a potato plant which is susceptible to Phytophthora infestans infections;
c) appropriate measures are taken so as to avoid mixed pollination;
d) seeds are isolated from the crop ripe fruits, if appropriate these are stimulated by appropriate measures to germinate, and are then sown;
e) plantlets are raised, and these are tested for resistance to Phytophthora infestans;
f) plants are selected which have the feature of resistance to Phytophthora infestans infections; and if appropriate these plants are obtained by periodically establishing a meristem culture.

### DEPOSITING

A Solanum tuberosum SCP-02 sample was deposited in the form of a meristem culture at the German Collection of Microorganisms and Cell Cultures GmbH (DSMZ) in 38124 Braunschweig, Mascheroder Weg 1b, dated 22 Apr. 2005 under deposit number DSM 17671.

### EXAMPLES

The following examples only serve to illustrate the present invention and are in no way restrictive for the scope of the invention.

### A. Plant Materials:

Commercial potato varieties [Potato Variety Adviser for Organic Farming http://www.uni-giessen.de/orgiandbau/moeller-sorten-beschreibung.htm]:

### Bintje:

This white-skinned, old variety is extremely susceptible both to viruses and to Phytophthora infestans,

Désirée:

Very susceptible to viruses. Fairly resistant, however, to P. infestans. Red-skinned.

### Sirtema:

Very susceptible to Phytophthora and viruses. White-skinned.

### Fontane:

The susceptibility of Fontane to P. infestans is moderate. However, health with regard to viruses is good. White-skinned.

### Agria:

Agria is moderately susceptible to P. infestans. It is very resistant to PVY and PLRV. White-skinned.

### Agata:

A new variety which is fairly resistant to viruses, but is susceptible to P. infestans. White-skinned.

### Blue Congo:

An old variety which is also called Blue Swede and the origin of which is not known. Probably the only wild variety which is cultivated in Central Europe. Nothing is known regarding susceptibility to leaf blight and viruses. The skin and flesh are purple/blue.

Within the framework of the following examples frequently cultivated standard varieties were used; the Blue Congo variety is included as a wild variety. The potatoes were not cropped, but were reused as tubers.

### B. Potatoes From Test Systems:

In 1953, B. LACK et al. introduced the test range which is still recognised and used internationally today (Tab. 1). It contains a total of 11 R genes on their own and in combination, however it can no longer be propagated by seed (information from the Biological Federal Institute for Agriculture and Forestry, Berlin-Dahlem, “Potato Leaf and Tuber Blight and its pathogen Phytophthora infestans (Mont.) De BARY”, Bärbel Schöber-Butin, issue 384, Berlin 2001, published by: Biological Federal Institute for Agriculture and Forestry, Berlin and Braunschweig; Parey Buchverlag Berlin).

With the help of the test range, as already described, the pathotypes of the fungus can be determined. Conversely however, the main genes which may be present in the potatoes can also be characterised with the help of individual pathotypes.

TABLE 1
Composition of the international Test Range (origin:
Scottish Agricultural Science Agency)

| R gene | potato variety/line |
|---|---|
| R | Craigs royal |
| R1 | Craigs snow white |
| R2 | 1512 |
| R3 | Pentland Ace |
| R4 | 1563c |
| R5 | 3053-18 |
| R6 | D2-21 |
| R7 | 218ef |
| R8 | 2424a |
| R9 | not known |
| R10 | 3861ad |
| R11 | 5008ab |

### Dutch Potato Lines (DPL):

The Dutch Lines are a test system for 11 Bintje plants. Every plant of the system has one of the R genes from Table 1. In this way the virulence genes of a fungus can be determined. The plantlets are propagated in tissue cultures, and planted in soil as required.

### C. The SCP-02 Potato:

It was possible to isolate the SCP-02 potato from a test field with potatoes of the “Bintje” variety which was infected with P infestans. It was tested with the help of the DPL system for resistance to the known pathotypes of Phytophthora infestans. The SCP-02 potato proved to be resistant here to P. infestans. Compared to the different potato viruses it is very susceptible. It is very palatable (similar to Matilda and Bintje) and white-skinned. It is used both as a tuber and as a whole plant for tests.

### Agronomic Description:

In cold storage (5° C.) the tubers produce sprouts easily from January onwards, and from March onwards clear seedling growth starts which is slightly more pronounced than with the comparable variety “Bintje”.

Germination prior to planting out takes place for 1 week under very intense light.

The tubers which have been planted out produce the first shoot tips on the surface in a comparable time frame to the comparable variety “Bintje”.

The development time up to flowering is also identical to that of “Bintje”. However, the plant size is considerably smaller than with the standard variety “Bintje”.

The SCP02 blossom is white, the flowers adhere well to the bush and consequently also develop fruits without any problem.

The tests carried out were limited to culture times which are typical for table potatoes (100-120 days). Suitability as an early potato or an industrial potato was not tested.

Storability: Normal (up to March). When using germination retardants similar storage possibilities to those with the comparable variety “Bintje” can be implemented.

| | |
|---|---|
| Susceptibility to virus (leaf roll virus): | average to high |
| Susceptibility to Phytophthora leaf: | none |
| Susceptibility to Phytophthora tubers: | none |
| Susceptibility to Alternaria | average |
| Attractiveness for aphids | average |
| Attractiveness for potato beetles | low |

Further details relating to the eating quality of SCP-02 in comparison with the high-performance varieties “Bintje” and “Matilda”, differentiated according to the cooking type, can be found in Table 2 below.

General Criteria for Assessing the Cooking Type of Potatoes

| Criteria/Evaluation | 1 | 2 | 3 | 4 |
|---|---|---|---|---|
| cooked | remains whole | slight splitting | pronounced | cooked to rags |
| | | | splitting | |
| consistency of the | firm | moderately firm | fairly soft | soft with uneven |
| flesh | | | | consistency |
| flouriness | not floury | slightly floury | floury | very floury |
| moisture | moist | slightly moist | fairly dry | dry |
| grain structure | fine | fairly fine | fairly coarse | coarse |
| colour | W 1 = pure white | W 2 = grey/white | W 3 = yellowy | |
| | Y 1 = light yellow | Y 2 = yellow | white | |
| | | | Y 3 = dark yellow | |
| flavour | none | weak | strong | very strong |

TABLE 2
Assessment of the Eating Quality of Potatoes According to the Cooking Type

| | Sample No. | | | |
|---|---|---|---|---|
| | 1 | 2 | 3 | 4 |
| line/variety | Syngenta-SCP-02-1 | Syngenta SCP-02-2 | Matilda | Bintje |
| cooked | 2 | 2-1 | 1-2 | 3 |
| consistency of flesh | 2 | 2 | 2-3 | 3-2 |
| flouriness | 2-3 | 2 | 1-2 | 3-2 |
| moisture | 2 | 2 | 1-2 | 3-2 |
| grain structure | 2-3 | 2 | 2 | 3 |
| cooking type | B-C | B | B-(A) | C-B |
| colour | Y2 | Y2 | Y2 | Y2 |
| flavour | pleasant | pleasant-slight | slight-pleasant | strong-pleasant |
| comments (after-taste etc.) | slightly grey | | sweet, insipid | |

cooking type:
A (overall pos. 1) = firm salad potato
B (overall pos. 2) = fairly firm potato suitable for all purposes
C (overall pos. 3) = floury potato
D (overall pos. 4) = very floury potato

### D. Phytophthora Strains:

Within the framework of the infection studies described below, the following Phytophthora strains were used:

### Strain 96:

Standard strain with virulence genes 3,4,5,7 and 9. [according to comparison with the Dutch lines]

### Strain 90128

Strain from Ohio with virulence genes 1,3,4,7,8,9,10 and 11.

### Strain 91002

Strain from the Netherlands with virulence genes 2 and 7.

### Strain 91011

Strain from the Netherlands with virulence genes 3,4,5 and 11.

### Strain 88069S

Strain from Ohio with virulence genes 1,3,4,7.

(The above-specified strains can be obtained from the University of Wageningen, Francine Govers Laboratory of Phytopathology, Wageningen Agricultural University, Binnenhaven 9, 6709 P D Wageningen, The Netherlands; Graduate School Experimental Plant Sciences, The Netherlands)

Alternatively to this, the different pathotypes of Phytophthora infestans can also be determined with the help of the test range of LACK et al. 1953 (Tab. 1).

A strain mixture with 230 isolates according to I. Irzhansky and Y. Cohen, 2006 (“Inheritance of resistance against Phytophthora infestans in Lycopersicon pimpenellifolium L3707”, accepted for publication in Euphytica) was used in order to gain further expertise on resistance in F1 progeny. Samples of the aforementioned strain mixture can be obtained from the authors.

All strains of fungus are cultivated on rye agar [Beyer K. et al, Characterization of Phytophthora infestans genes regulated during the interaction with potato. Molecular Plant Pathology 2002, 3(6), 473-85].

### Example 1

### Determining Relationships

In order to determine the relationships between organisms there are various possibilities which are based e.g. upon the use of Simple Sequence Repeats (SSR), Random Amplified Polymorphic DNA-Polymerase-Chain-Reaction (RAPD-PCR) and Amplified-Fragment-Length-Polymorphism (AFLP). For all four methods DNA must first of all be extracted, purified and quantified. Within the framework of the present invention the relationship was determined by means of AFLP (G. Knapova and U. Gist Phenotypic and genotypic structure of Phytophthora infestans populations on potato and tomato in France and Switzerland, Plant Pathology Volume 51, Issue 5, Page 641—October 2002).

The AFLP technique is based upon amplification of the products of restriction digestion and subsequent selecting PCR. The products produced by these reactions are then loaded onto a polyacrylamide gel and separated for 4 hours at high voltage.

The DNA fragments marked “in colour” by the selective PCR flow through a sensor at the bottom end of the gel which registers their arrival. All of the recordings combined then produce the band diagram which is evaluated.

The genetic finger print of the Phytophthora-resistant SCP-02 potatoes is not similar to any of the commercial varieties to such an extent that it could have been produced by point mutation of the latter. Therefore, SCP-02 is not closely related to any of the tested varieties from Central Europe. By far the greatest similarity is shown by SCP-02 (as can be seen in Table 1) with Désirée, and the least with the wild variety Blue Congo (=Blue Swede). Table 1 is a summary of the binary evaluation according to the number of corresponding band features.

TABLE 2

| | | | | Sir- | Fon- | | | |
|---|---|---|---|---|---|---|---|---|
| | SP02 | Bintje | Désirée | tema | tane | Agata | Agria | Swede |
| SP02 | | 120 | 145 | 131 | 137 | 132 | 128 | 118 |
| Bintje | 120 | | 132 | 112 | 120 | 133 | 129 | 113 |
| Désirée | 145 | 132 | | 127 | 143 | 146 | 140 | 126 |
| Sirtema | 131 | 112 | 127 | | 129 | 136 | 124 | 120 |
| Fontane | 137 | 120 | 143 | 129 | | 140 | 142 | 128 |
| Agata | 132 | 133 | 146 | 136 | 140 | | 141 | 129 |
| Agria | 128 | 129 | 140 | 124 | 142 | 141 | | 137 |
| Swede | 118 | 113 | 126 | 120 | 128 | 129 | 137 | |

### Example 2

### Infection Stages

The infection pattern was investigated by means of the resistant potato SCP-02 and a “Bintje” as a control plant. In so doing, the tubers of the two potato plants were stored for ten weeks at a temperature of 4° C. and germinated by forming stolons. The plantlets were then raised from the stolons.

For the infection studies 2 week old Phytophthora strains (strain 96 and strain 90128) were used so as to guarantee good sporulation.

For the preparation test three leaf segments of SCP-02, Bintje and a self-fertilised SCP-02 F1-hybrid plant are respectively placed with moist filter papers in petri dishes with the bottom of the leaf facing upwards and are inoculated with a spore suspension of the two fungus strains. The dishes are incubated for one day in the dark and then for four days in the light. After five days the leaves are visually assessed for the resistance type (resistant, sensitive or vertically resistant).

The spore density is 50,000 spores/ml. For optimal zoo spore release the suspension is cooled for at least 30 minutes at 4° C. The spore suspension is now sprayed onto the lower side of the leaf and the leaf is incubated. After one day one leaf respectively is cut from each combination, boiled in the heated dye solution and incubated for one day in the latter. On the following day the leaf is decoloured and observed under the microscope. The other two leaves are correspondingly evaluated on the third and on the fifth day. The observations are documented both in text and in picture form and compared.

With the colouring no indications were shown of increased infection. The zoo spore germinated, formed a germ tube and an appressorium, by then died back very quickly. Therefore, the fungus can only penetrate into the surface of the leaf, but can not trigger any infection here (no mycelium or spore formation). The sensitive and the vertically resistant plants showed the expected infection pattern.

### Example 3

### Resistance Mechanisms

In order to differentiate between different possible resistance mechanisms (horizontal resistance or vertical resistance) in SCP-02, the potato must be tested with highly virulent Phytophthora strains. In order to rule out that the resistance of SCP-02 may be based upon an R gene, leaf segments and potato slices were first of all inoculated with Phytophthora strains which cover the majority of the R genes of the DPL. For this, leaf segments and the washed potato slices were placed in petri dishes which were provided with moist filter papers, and 10 droplets of the cooled spore suspension (50,000 spores/ml) were pipetted onto the lower side of the leaf or the slices. Next, the dishes were incubated for five days and then analysed for symptoms. The same test was also carried out with highly virulent strains from Switzerland and from Israel (standard strain of the Bar-Ilan University, Israel).

Over several years field cultivation in locations with natural Phytophthora inoculum SCP-02 always remained free of infection symptoms both on the leaves and on the tubers. In the tests carried out here SCP-02CP-02 also proved to be resistant to all previously tested Phytopthora infestans pathotypes. The pathotypes which could affect the differential potato lines (R1-R11, R6 had not yet been tested) were not able to trigger any infection with SCP-02. Also with inoculation with a strain mixture of more than 200 isolates SCP-02 remained symptom-free.

By determining the stopping time of the pathogen during the infection, the stage can be determined fairly accurately. The defence reaction seems to start directly after formation of the appressorium. The fungus does not penetrate, and under the microscope no cells destroyed by a hypersensitive reaction (HR) can be seen. These factors lead to the conclusion that the resistance can not be vertical, but that horizontal resistance brought about polygenically is present in the plant. That the fungus can not penetrate the cuticula could be due to the composition of the cutin. If this is slightly different than with conventional potato varieties, it can not be broken down by the pathogen cutinase. It could be, therefore, that this is a “non-host” reaction (the pathogen does not recognise the SCP-02 as a host plant). Since the resistance is caused polygenically, however, other such factors (e.g. lack of receptors on the leaf surface) and mechanisms are conceivable. The error source with this test is very small because the coloured leaves can be observed very well under a well-equipped microscope.

If on the other hand, one inoculates leaf segments or potato slices with the highly pathogenic Phytophthora strain 90128, a different reaction will be observed. The typical symptoms of a Phytophthora infection will be seen. The leaves become watery, have a bad smell and slowly decompose—all symptoms which are typical of a Phytophthora infection. Unlike an infection which triggers leaf and root blight, in the case of the SCP-02 potato however no mycelium is formed on the surface of the lower side of the leaf and the fungus does not sporulate.

A similar picture occurs with a tuber infection. Mycelium is formed on the tuber. This grows in a ball shape, however, and does not sporulate. Accordingly, the fungus can indeed penetrate into the surface of the plant, but can not propagate any further and not release any infection which causes disease.

### Example 4

### Resistance Heredity

### 4.1 Blossom Induction:

The potato plants were stimulated to produce more blossom by the following method. The seed corm was placed on a wooden beam covered with soil, and more soil was sprinkled over the top. The plant grew over the beam, including the roots and the tubers. The tubers formed were removed monthly without damaging the plants excessively. The “beam plants” flowered more abundantly than the controls.

### 4.2 Self-Pollination

Since the potato is capable of self-pollination, freshly opened flowers of the SCP-02 plant, the anthers of which were not yet open, and older flowers from a commercial variety (Desiree and Fontane) were used for the heredity tests.

With the actual crossing, first of all the anthers of the maternal plant were cut out of the flowers and then, using a preparation needle, pollen from the paternal plant was transferred to its stigma. The pollinated blossom was placed in a small paper bag so as to prevent mixed pollination. After approximately eight weeks the fruits which had formed were ripe for cropping. In addition to the crossed fruits, self-pollinated fruits were also harvested in order to compare the two heredities. The berries were cut out and incubated in their own sap for two days in order to increase the germination capacity of the seeds. After this, the seeds were sown and the plantlets raised. As soon as the plantlets were large enough (after approximately eight weeks), they were tested for their resistance to Phytophthora infestans (according to the method described above). The respective number of plants with the different resistance features is given as a ratio and then compared with the existing ratios.

The F1 plants can be obtained by storing the tubers formed or by establishing meristem cultures.

### 4.3 Cross Analysis

If one tests the progeny produced by the cross for its resistance properties, one sees a division of the phenotypes into three groups. Resistant phenotypes are obtained comparable to SCP-02, phenotypes which show a (H)ypersensitive (R)eaction, and sensitive phenotypes. The ratio of these phenotypes when SCP-02 is crossed with Fontane produced a division of 6:58:1 (resistant:HR:sensitive). This leads one to conclude that these are tetraploid crossing partners. The F1 plants produced by self-pollination are all resistant to strain 96. However, the morphology can differ greatly. Upon the basis of the self-pollination test it can be ruled out that the resistance is inherited recessively so that one can assume that there is dominant heredity.

With the SCP-02×Desiree cross there was a 2:5:0 (resistant:HR:sensitive) division.

Inoculation of 12 randomly selected representatives of F1, all of which however included F1 resistant to strain 96, with a strain mixture of 230 isolates according to I. Irzhansky and Y. Cohen, 2006, produced segregation of 4:5:3 (resistant:HR:sensitive).

The resistant F1 progeny from the SCP-02×Fontane and SCP-02×Desiree cross were then inoculated with the strain 90128. Of 2 Desiree progeny one remained resistant, and of 6 Fontane progeny 6 remained resistant, i.e. they showed total immunity to all of the Phytophthora strains tested.

The segregation (6:58:1) of the F1 progeny of the SCP-02×Fontane cross leads one to conclude that the parents are tetraploid. Since the progeny resulting from the self-pollination are all resistant to strain 96, it is assumed that the main resistance factor is inherited dominantly. However, the test with further pathogen strains showed that resistance has further factors beyond HR because the resistant F1 progeny divided again. It is certain, therefore, that several resistance mechanisms are at work in SCP-02. This is no longer remarkable if one examines the genetics of autotetraploids; the pollen of these plants can have 6¹² different allele combinations. However, upon the basis of the heredity diagram it can be said that when a commercial variety is crossed with SCP-02, totally resistant plants are always produced. The strain mixture of 230 isolates with all virulences and a wide range of aggressivities could not affect all F1 either, and this suggests that total resistance can be stably inherited. The results with self-pollination tests show that the resistance heredity runs dominantly (all F1s are resistant, but different in phenotype) and SCP-02 is a heterozygote. The result of these two tests once again supports the assumption that the resistance is brought about polygenically. The crossing produced very many vertically resistant plants with which due to the crossing at least one resistance factor was “lost”, i.e. is recessive.

According to the results achieved, it is possible to cultivate a potato variety from SCP-02 by means of combination cultivation which is resistant in the long-term to Phytophthora infestans and also provides a good yield. Horizontal resistance, crossing capability and dominant resistance heredity are ideal basic conditions for this.

On the other hand, SCP-02 can be used as a donor for resistance to Phytophthora infestans in cases where crossing is difficult or impossible (in Solanum tuberosum spp. andigena, but also with other Lycopersicon types). For this, the genetic factors which lead to resistance must be determined with further studies.

### 4.4 Propagating and Obtaining the F1 Generations by Means of Meristem Cultures

Sprout tips are cut from the potato plant such that at least 2 nodes remain on the explant.

The sprout tips are surface-sterilised under sterile conditions in a 0.5% hypochlorate solution (( NaHClO)=Javel water) (for approx. half a minute) and then bathed with sterile tweezers in sterile water. The explants are then cultivated on sterilised Murashige & Skoog medium stiffened with agar (Murashige Mix SIGMA 4.3 g/l; saccharose 15 g; agar 5 g; E water 1000 ml). The sprout tips are arranged on the medium here such that the two uppermost nodes extend into the medium and are surrounded by the latter.

The explants prepared in this way are cultivated in the climate chamber at a daytime temperature of 22° C. and a night-time temperature of 20° C. and with relative humidity of 60% and a day/night rhythm of 16 hrs/8 hrs.

After approx. 10 days the first roots become visible.

1. A potato plant which has horizontal, and so outstanding and lasting resistance to Phytophthora infestans infections and preferably remains free of symptoms.

2. The potato plant according to claim 1, characterised in that the infection is one caused by Phytophthora infestans pathotypes which contain at least one or more of the virulence genes selected from the group of virulence genes 1, 2, 3, 4, 5, 7, 8, 9, 10 and 11 in different combinations.

3. The potato plant according to claim 1, characterised in that the infection is one caused by Phytophthora infestans pathotypes which contain two, three, four, five, six, seven, eight, nine, ten or in particular all of the virulence genes selected from the group of virulence genes 1, 2, 3, 4, 5, 7, 8, 9, 10 and 11 in different combinations.

4. The potato plant according to claim 3, characterised in that the infection is one caused by one or more of the following Phytophthora infestans pathotypes, in particular however by all of the following pathotypes which have at least 2 of the known virulence genes V1-V11 in the following combinations
Pathotype 1: with virulence genes 3, 4, 5, 7 and 9
Pathotype 2: with virulence genes 1, 3, 4, 7, 8, 9, 10 and 11
Pathotype 3: with virulence genes 2 and 7
Pathotype 4: with virulence genes 3, 4, 5 and 11
Pathotype 5: with virulence genes 1, 3, 4, 7

5. The potato plant according to claim 1, characterised in that the resistance feature can be obtained by the hybridisation of a precursor plant which has the genetic form of SCP-02 (DSM 17671).

6. The potato plant according to claim 1, which includes the feature of resistance to Phytophthora infestans infections, hybridised from a precursor plant which has the genetic form of SCP-02 (DSM 17671).

7. The potato plant according to claim 1, characterised in that when infected with a highly pathogenic Phytophthora infestans strain, the aforementioned resistance feature is characterised in that the natural infection pattern of Phytophthora infestans is interrupted in that the formation of mycelium on the lower side of the leaf and sporulation are prevented and the infection symptoms therefore remain restricted to the location of the infection.

8. A method for the hybridisation of resistance to infections with Phytophthora infestans in potato plants which do not have this resistance, characterised in that
a) a potato plant which has the genetic form of SCP-02, and so resistance to Phytophthora infestans infections, a plant from which a sample in the form of a meristem culture has been deposited at the German Collection of Microorganisms and Cell Cultures GmbH (DSMZ) in 38124 Braunschweig, Mascheroder Weg 1b, dated 22 Apr. 2005 under deposit number DSM 17671, is crossed with a potato plant which is susceptible to Phytophthora infestans infections;
b) appropriate measures are taken so as to avoid mixed pollination;
c) seeds are isolated from the crop ripe fruits and sown;
d) plantlets are raised, and these are tested in an appropriate test system for resistance to Phytophthora infestans;
e) plants are selected which have the feature of resistance to Phytophthora infestans infections.

9. The method according to claim 8, characterised in that the F1 hybrids resulting from the crossing and which have the feature of resistance to Phytophthora infestans infections, are re-crossed between one and more times with a parent.

10. The method according to claim 9, characterised in that the aforementioned parent is the Phytophthora-sensitive parent.

11. The method according to claim 10, characterised in that a plant is obtained which has between 80% and 99.5%, better between 90% and 99%, but in particular between 95% and 98% of the genome of the sensitive parent plant, but is moreover still characterised by the feature of resistance.

12. The method according to claim 10, characterised in that the sensitive parent is a high-performance variety.

13. A method for producing a potato plant which is resistant to Phytophthora infections, characterised in that
a) a potato plant which has the genetic form of SCP-02, and so resistance to Phytophthora infestans infections, a plant from which a sample in the form of a meristem culture has been deposited at the German Collection of Microorganisms and Cell Cultures GmbH (DSMZ) in 38124 Braunschweig, Mascheroder Weg 1b, dated 22 Apr. 2005 under deposit number DSM 17671, is crossed with a potato plant which is susceptible to Phytophthora infestans infections;
b) appropriate measures are taken so as to avoid mixed pollination both during and after the artificially implemented pollination process;
c) seeds of the F1 generation are isolated from the crop ripe fruits and sown;
d) plantlets are raised, and these are tested for resistance to Phytophthora infestans;
e) Phytophthora-resistant plants are re-crossed between one and more times with the Phytophthora-susceptible parent;
f) in each re-crossed generation obtained in this way, the plants are tested for the presence of absence of the resistance feature;
g) a plant is raised which is characterised by the desired feature of resistance to Phytophthora infections.

14. A method for the hybridisation of resistance to infections with Phytophthora infestans in potato plants which do not have this resistance, characterised in that
a) if appropriate the potato plant provided as the mother plant is stimulated by appropriate measures to form blossom;
b) a potato plant which has the genetic form of SCP-02, and so resistance to Phytophthora infestans infections, a plant from which a sample in the form of a meristem culture has been deposited at the German Collection of Microorganisms and Cell Cultures GmbH (DSMZ) in 38124 Braunschweig, Mascheroder Weg 1b, dated 22 Apr. 2005 under deposit number DSM 17671, is crossed with a potato plant which is susceptible to Phytophthora infestans infections;
c) appropriate measures are taken so as to avoid mixed pollination;
d) seeds are isolated from the crop ripe fruits, if appropriate these are stimulated by appropriate measures to germinate, and are then sown;
e) plants are raised, and these are tested for resistance to Phytophthora infestans;
f) plants are selected which have the feature of resistance to Phytophthora infestans infections; and if appropriate, these plants are obtained by periodically establishing a meristem culture.
